(19) United States
(12) Patent Application Publication
(10) Pub. No.: US 2007/0048244 A1
(43) Pub. Date: Mar. 1, 2007

(54) Cosmetic composition for cleaning the skin

(75) Inventor: Tyzz-Chuan Tseng, Taipei (TW)

Correspondence Address: COOPER & DUNHAM, LLP, 1185 AVENUE OF THE AMERICAS, NEW YORK, NY, 10036, US

(73) Assignee: Feng-Ching O

(21) Appl. No.: 11/499,221

(22) Filed: Aug. 4, 2006

(30) Foreign Application Priority Data
Aug. 26, 2005 (TW) 094129167

(51) Int. Cl. A61K 8/64; A61K 8/97; A61K 36/06

(52) U.S. Cl. 424/74; 424/195.15; 424/70.14

(57) ABSTRACT

A cosmetic composition for cleansing skin has Ganoderma sp. spores in the amount of about 15% to about 50% (w/w) of the composition and collagen in the amount of about 50% to about 85% (w/w) of the composition.

### BACKGROUND OF THE INVENTION

1. Field of the Invention

The present invention relates to a cosmetic composition for cleansing the skin, and especially to a cosmetic composition including Ganoderma sp. spores for cleansing the skin.

2. Description of the Related Art

A conventional chemical cleaning agent for facial shin includes lotions or soaps. Lotions and soaps contain surfactants, emulsifying agents, foaming agents, softeners, preservative, etc. Lotion or soap is rubbed on a person's face to emulsify and remove oil from the person's face.

The foregoing chemical cleansing technique removes large molecule emulsifying agents and oily substances because the large molecule emulsifying agents and oily substances can dissolve in small molecule emulsifying agents and be rinsed. However, calabash-like hair follicles and the sweat glands retain a little lotion, oily substances and ingredients of cosmetic compositions after cleansing the face and block the hair follicles and the sweat glands or are absorbed via the skin. Furthermore, the chemical cleansing technique cannot remove external, aging epidermis so the facial skin darkens and is detrimental to the complexion.

In addition, chemical substances may induce allergic reaction that is not comfortable for the user. When the skin is cleansed incompletely, the skin is burdened with retained chemical substances.

### SUMMARY OF THE INVENTION

The objective of the present invention is to provide a cosmetic composition comprising Ganoderma sp. spores and collagen to physically cleanse skin. Ganoderma sp. spores are small and oval-shaped, and each spore has a cell wall and multiple holes. The cell wall is composed of a large amount of chitin. The collagen has an adhesion force. After mixing Ganoderma sp. spores and the collagen well, a user applies the cosmetic composition to the skin. The Ganoderma sp. spores absorb oil and other substances in the hair follicles and the sweat glands. Then the collagen bonds the Ganoderma sp. spores, substances in the hair follicles and the sweat glands and the aging epidermis. In addition, the collagen in the present invention is in powder form so that growth of microbiological organisms is suppressed and preservatives are not need.

To achieve the foregoing objective, a cosmetic composition for cleansing the skin in accordance with the present invention comprises Ganoderma sp. spores in the amount of about 15% to about 50% (w/w) of the composition and collagen in the amount of about 50% to about 85% (w/w) of the composition.

Other objectives, advantages and novel features of the invention will become more apparent from the following detailed description when taken in conjunction with the accompanying drawings.

### DETAILED DESCRIPTION OF THE PREFERRED EMBODIMENTS

A cosmetic composition for cleansing skin in accordance with the present invention comprises a mixture of Ganoderna sp. spores, collagen and preferably water and perfumed oil to cleanse the skin.

The Ganoderma sp. spores are in an amount of about 15% to about 50% (w/w) of the composition, are oval-shaped spores of 8 μm in size, easily go into the hair follicles and the sweat glands that are 100 μm in size, may have an odor and have a cell wall and multiple holes. Preferably, the Ganoderma sp. spores are in the amount of about 15% to about 25% (w/w) of the composition. The cell wall of Ganoderma sp. spores is composed of double layers of chitin and has an outer surface. Preferably, the cell wall of Ganoderma sp. spores is unbroken. The holes are formed the in the outer surface much like dimples on the surface of a golf ball and absorb and hold oil and chemical substances from the hair follicles and the sweat glands.

The collagen is in an amount of about 50% to about 85% (w/w) of the composition, is preferably in powder form and is preferably extracted from fish skin. Preferably, the collagen is in the amount of about 75% to about 85% (w/w) of the composition. Furthermore, drying the collagen bonds the Ganoderma sp. spores, the oil and chemical substances in the hair follicles and the sweat glands and external, aging epidermis to cleanse the skin.

Water is added to the mixture to form a paste like mixture.

The perfumed oil inhibits the growth of bacteria and covers the odor of Ganoderma sp. spores. More preferably, the perfumed oil is lavender oil.

The term “unbroken” used in accordance with the present invention refers to a complete shape of the Ganoderma sp. spores without damage.

### EXAMPLE

1. Material

Unbroken Ganoderma sp. spores were bought from Da Han Biotechnology Company.

Powder form of collagen from fish skin was brought from First Chemical Engineering Company and imported from Japan.

Oil blotting paper was imported from Japan.

2. Preparation of Thick Collagen Mixture

After mixing the Ganoderma sp. spores and powder form of the collagen, water was added and mixed well to form a thick collagen mixture.

3. Step of Usage

After preliminarily cleansing the skin, the thick collagen was applied to the skin and an oil blotting paper was placed on the thick collagen mixture to form a mask-like application. Additional thick collagen mixture was applied to the oil blotting paper to secure the adhesion force. Preferably, the oil blotting paper and the thick collagen mixture are removed together with a ripping motion. After drying the thick collagen mixture for about ten minutes, the mask-like oil blotting paper with the collagen and Ganoderma sp. spores were removed from the skin with a ripping motion to remove oil and chemical substances in the hair follicles and the sweat glands and external, aging epidermis. Then any residual thick collagen mixture is rinsed away with water. When the thick collagen mixture dries, the thick collagen mixture bonds to the Ganoderma sp. spores and external, aging epidermis to cleanse the hair follicles and the sweat glands and remove the external, aging epidermis.

4. Result

4.1 The ratio of Ganoderma sp. spores and the collagen

| | | Ratio | | | | | |
|---|---|---|---|---|---|---|---|
| Ganoderma spores (w/w) | | 10 | 15 | 20 | 25 | 50 | 75 |
| Collagen (w/w) | | 90 | 85 | 80 | 75 | 50 | 25 |
| Result | Adhesive force^{a} | 1 | 2 | 3 | 4 | 5 | 6 |
| | Cleansing force^{b} | 5 | 2 | 1 | 3 | 4 | 6 |

^{a}adhesive force is arranged in order from major to minor.
^{b}cleansing force is arranged in order from major to minor.

Therefore, when the ratio of the collagen increases, the adhesive force increases. When the ratio of the collagen is greater than 80%, the adhesive force is very large so that ripping the collagen from the skin irritates the skin. In addition, the ratio of Ganoderma sp. spores is very low so that the effect of cleanness reduces. On the contrary, when the ratio of the collagen is too low, the adhesion force of the collagen reduces so that the collagen cannot bond the Ganoderma sp. spores and the substances and oil in the hair follicles and the sweat glands. Therefore, the ratio of the collagen in the cosmetic composition is from about 50% (w/w) to about 85% (w/w) and the ratio of the Ganoderma sp. spores in the cosmetic composition is from about 15% (w/w) to about 50% (w/w).

Therefore, the ratio of the collagen in the cosmetic composition is from about 50% (w/w) to about 85% (w/w) and the ratio of the Ganoderma sp. spores in the cosmetic composition is from about 15% (w/w) to about 50% (w/w). The collagen bonds well and the cleansing effect is good. Preferably, the ratio of the collagen in the cosmetic composition is from about 75% (w/w) to about 85% (w/w) and the ratio of the Ganoderma sp. spores in the cosmetic composition is from about 15% (w/w) to about 25% (w/w).

4.2 Test

Number of tests: 120 people

Method: Water was added to the equal amounts of Ganoderma sp. spores and powder form of collagen and mixed well to form thick collagen mixture. The thick collagen mixture was applied to the facial skin and oil blotting paper was applied to the thick collagen mixture. Then more thick collagen mixture was applied to the oil blotting paper. After about ten minutes, the drying thick collagen and oil blotting paper were ripped off the facial skin, and the facial skin was rinsed.

| Smooth | Number |
|---|---|
| Very smooth | 115 |
| Smooth | 5 |
| Slightly smooth | 0 |
| No change | 0 |

This shows that the cosmetic composition in accordance with the present invention has a good cleansing effect.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A cosmetic composition for cleansing skin comprising a mixture of
Ganoderma sp. spores in the amount of about 15% to about 50% (w/w) of the composition; and
collagen in the amount of about 50% to about 85% (w/w) of the composition.

2. The cosmetic composition as claimed in claim 1, wherein Ganoderma sp. spores are in the amount of about 15% to about 25% (w/w) of the composition.

3. The cosmetic composition as claimed in claim 1, wherein the cell wall of Ganoderma sp. spores is unbroken.

4. The cosmetic composition as claimed in claim 1, wherein the collagen is in powder form.

5. The cosmetic composition as claimed in claim 1, wherein the collagen is in the amount of about 75% to about 85% (w/w) of the composition.

6. The cosmetic composition as claimed in claim 1, wherein the collagen is extracted from fish skin.

7. The cosmetic composition as claimed in claim 1, wherein the cosmetic composition further comprises water for making the cosmetic composition become glue-like.

8. The cosmetic composition as claimed in claim 1, wherein the cosmetic composition further comprises perfumed oil.
